(19) United States
(12) Patent Application Publication
(10) Pub. No.: US 2005/0228417 A1
(43) Pub. Date: Oct. 13, 2005

(54) Devices and methods for removing a matter from a body cavity of a patient

(76) Inventors: George P. Teitelbaum, Santa Monica, CA (US); Donald W. Larsen, La Canada, CA (US)

Correspondence Address: HOGAN & HARTSON L.L.P., 500 S. GRAND AVENUE, SUITE 1900, LOS ANGELES, CA, 90071-2611, US

(21) Appl. No.: 11/087,780

(22) Filed: Mar. 23, 2005

Related U.S. Application Data
(60) Provisional application No. 60/556,993, filed on Mar. 26, 2004.
(60) Provisional application No. 60/611,684, filed on Sep. 20, 2004.

(51) Int. Cl. A61B 17/22

(52) U.S. Cl. 606/159

(57) ABSTRACT

Disclosed are devices for removing a matter from a body cavity of a patient. One of such devices has an elongated carrier having a distal portion adapted to move through or within the cavity and a proximate portion. A radially expandable polymer is circumferentially attached to the distal portion of the carrier and adapted to enter the matter while in a compressed configuration and capable of transitioning to an expanded configuration while inside the matter to penetrate and engage it from within. Methods of removing a matter from a body cavity and methods of localized delivery of a therapeutic agents also are disclosed.

This application claims priority to the U.S. Provisional Patent Application No. 60/556,993, filed on Mar. 26, 2004, and the U.S. Provisional Patent Application No. 60/611,684, filed on Sep. 20, 2004.

### FIELD OF THE INVENTION

This invention relates to devices and methods for removing a matter from a body cavity of a patient and delivery of a therapeutic agent. In particular, the invention is directed to devices, including, but not limited to, endovascular devices, comprising a radially expandable polymer for engaging and removing the matter.

### BACKGROUND OF THE INVENTION

A number of vascular disorders, such as stroke, pulmonary embolism, peripheral thrombosis, and atherosclerosis, are characterized by formation of occlusions that prevent normal blood flow in blood vessels. For example, an ischemic stroke is a neurological dysfunction caused by a blockage of one of the major arteries of the brain. The blockage can be the result of the formation of a blood clot at the site of blockage (thrombosis), obliteration of the lumen of a blood vessel caused by atherosclerosis, or the migration of an occluding blood clot (formed in the heart, carotid artery, or elsewhere) downstream to the site of blockage (embolization).

Clot-busting (thrombolytic) drugs have been employed to break up clots blocking a particular blood vessel. But the success rate of this approach is still very low. For example, at present, the only FDA-approved thrombolytic drug for acute (less than three hours old) ischemic stroke is tissue plasminogen activator (tPA). With this form of therapy, only 30% of patients are expected to realize a good or excellent clinical outcome several months following infusion, and patients who demonstrate signs of intracranial hemorrhage at the time of presentation (on a CT study of their heads) are not candidates for tPA therapy. Also, intravenous tPA therapy is associated with an almost 6% fatal intracranial hemorrhage rate. Because of these shortcomings, there has been increasing interest in the development of a mechanical means of clot retrieval or dissolution.

Concentric Medical, Inc. (located in Mountain View, Calif.) has created an intraluminal clot retrieval system consisting of a nitinol-(Nickel-Titanium alloy) shape-memory corkscrew-like coil that is advanced into an occluding clot (U.S. Pat. Nos. 5,895,398; 6,638,245; 6,530,935; and 6,692,509). The coil and its attached wire are then withdrawn from the affected cerebral vessel, retrieving the thrombus material into a balloon-tipped guiding catheter positioned in the internal carotid artery. This device has been shown, in a prospective nonrandomized human clinical study (MERCI Trial), to achieve a 53.5% revascularization rate, with a serious device and/or procedure-related adverse event rate of 7%. There was a 31% death rate in the recanalized patients versus a 57% death rate in the nonrecanalized patients. There was an 8% symptomatic intracerebral hemorrhage rate (lower than the 10% intracranial hemorrhage rate experienced during the intra-arterial thrombolysis PROACT II trial).

Although the results are promising, Concentric Medicals clot retrieval device suffered from an approximately 6% wire breakage rate. Thus, an unfulfilled need still exists for more reliable, safe, and effective mechanical clot retrieval devices. More generally, there is a need for reliable, safe, and effective devices and methods of retrieving a matter from a body cavity of a human or an animal patient.

### SUMMARY OF THE INVENTION

Accordingly, one object of the present invention is to provide devices and methods for engaging and removing a matter from a body cavity of a patient, including endovascular devices and methods for removing a matter from a lumen of a blood vessel. Another object of the invention to provide devices and methods for delivery of therapeutic agents.

These and other objects are achieved in the device of the present invention. The device comprises an elongated carrier having a distal portion adapted for positioning inside a body cavity and a proximate portion. A radially expandable polymer is circumferentially attached to the distal portion of the carrier and adapted to enter a matter located inside the body cavity while in a compressed configuration. The expandable polymer is capable of transitioning to an expanded configuration while inside the matter to penetrate and engage it from within.

The body cavity may be a naturally existing or surgically made conduit or cavity. Examples of such conduits and cavities include, but are not limited to, blood vessels; parts of the alimentary tract, including esophagus, stomach, small and large bowels, anus and rectum; parts of the genitourinary system, including renal pelvis, ureter, urethra, spermatic cord, fallopian tubes; the ventricles and cisterns of the brain; the urinary bladder; cysts, vagina; uterine cavity; pseudocysts; abcesses; and fistulae.

The transition of the polymer between the compressed configuration and the expanded configuration may be triggered by a physiological or an external stimulus. Examples of the physiological stimulus include, but are not limited to, body temperature, blood pH, an ion concentration in blood, and blood composition. Examples of the external stimulus include, but are not limited to, changes in the local chemical environment, changes in the external temperature, light, magnetic field, ultrasound, radiation, and electrical field. For example, a biocompatible solution may be introduced into the blood vessel that causes changes in the local chemical environment and results in the expansion of the polymer.

In one embodiment, the polymer is a hydrogel, and the transition of the hydrogel into the expanded configuration is triggered by a hydration of the hydrogel or by application of a triggering fluid to the hydrogel. In another embodiment, the polymer is a shape memory foam. For example, the shape memory foam may have an original expanded configuration that is compressed at a temperature above a glass transition temperature, Tg, to form the compressed configuration. The foam retains its compressed configuration at a temperature below Tg but returns substantially to its original expanded configuration when it is exposed to a temperature above the Tg.

The polymer in its expanded configuration may have any shape and form as long as the shape and form allow it to penetrate and engage the matter to be removed from within. For example, it may be in a form of a coil, a twisted ribbon, a screwlike structure, a disk, a sphere, a parachute-like structure, a formation comprising a plurality of ridges and troughs, and a formation comprising a plurality of outwardly extending spears. In one embodiment, the expanded configuration of the polymer has a twisted ribbon shape, and the polymer is capable of storing torque energy and releasing it on demand.

In another aspect, the present invention provides another device for retrieving a matter from a body cavity of a patient. The device comprises an elongated carrier having a distal portion adapted for positioning inside the body cavity and at least two isolated formations of radially expandable polymer attached to the distal portion of the carrier. Each formation encloses the entire circumference of the carrier. The formations are adapted to move through or around the matter while having a compressed configuration and capable of transitioning to an expanded configuration to trap the matter there between. In one embodiment, the polymer is a hydrogel or a foam. The device may comprise a plurality of progressively decreasing in size formations of the radially expandable polymer. For example, the formations may be disks, spheres, outwardly extending spears, or configurations comprising a plurality of ridges and troughs.

In still another aspect, the present invention provides another device for retrieving a matter from a body cavity of a patient. The device comprises an elongated carrier having a distal portion adapted for positioning inside the body cavity and a radially expandable polymer circumferentially attached to the distal portion of the carrier. The polymer is adapted to move through or around the matter while having a compressed configuration and is capable of transitioning to an expanded configuration to engage the matter for retrieval from the body cavity. In this embodiment of the invention, the transition of the polymer is triggered by a physiological stimulus.

In yet another aspect, the present invention provides a device with a retrieval element. The retrieval element is adapted for positioning inside a body cavity of a patient. The retrieval element has a proximal end and a distal end, comprising an expandable sleeve. The retrieval element has a channel that extends through the entire length of the retrieval element and the expandable sleeve. The retrieval element further includes an inflatable balloon positioned concentrically inside the channel. The balloon, when inflated, is capable of radially expanding the expandable sleeve. The device also includes an elongated carrier slidably positioned within the channel of the retrieval element, wherein the elongated carrier has a distal portion adapted to move through the expandable sleeve of the retrieval element into the body cavity. The device also has a radially expandable polymer circumferentially attached to the distal portion of the carrier and adapted to move through or around the matter while having a compressed configuration and capable of transitioning to an expanded configuration to engage the matter. In its expanded configuration the expandable polymer is capable of being at least partially retrieved into the expandable sleeve.

The invention also provides a number of methods of retrieving a matter from a body cavity of a patient. In one embodiment, the method comprises (a) providing a device having a carrier with a distal portion and a radially expandable polymer circumferentially attached to the distal portion of the carrier, wherein the expandable polymer has an initial compressed configuration; (b) advancing the distal portion of the carrier of the device into the body cavity; (c) positioning the expandable polymer inside the matter; (d) allowing a sufficient time for the expandable polymer to transition from the initial compressed configuration to an expanded configuration thereby penetrating and engaging the matter from within; and (e) retrieving the device from the body cavity, thereby removing the matter.

In another embodiment, the method comprises (a) providing a device having a carrier with a distal portion and at least two isolated formations of radially expandable polymer attached to the distal portion of the carrier, wherein each formation encloses the entire circumference of the carrier and the expandable polymer has an initial compressed configuration; (b) advancing the distal portion of the carrier into the body cavity; (c) passing at least one formation through or around the matter; (d) allowing sufficient time for the expandable polymer to transition from the initial compressed configuration to an expanded configuration, thereby trapping the matter between the formations; and (d) retrieving the device, thereby removing the matter.

In still another embodiment, the method of retrieving a matter from a body cavity of a patient comprises (a) providing a device having a radially expandable polymer circumferentially attached to the distal portion of the carrier, wherein the expandable polymer has an initial compressed configuration; (b) positioning the distal portion of the carrier inside the body cavity and through or around the matter; (c) allowing sufficient time for a physiological stimulus to act on the expandable polymer to cause its transition from the initial compressed configuration to an expanded configuration, thereby engaging the matter in a way that allows its removal; and (d) retrieving the device, thereby removing the matter.

In yet another aspect, the invention provides a method of localized delivery of a therapeutic agent. The method comprises (a) providing a removable device having a carrier with a distal portion and a radially expandable polymer circumferentially attached to the distal portion of the carrier, wherein the expandable polymer has an initial compressed configuration; (b) advancing the distal portion of the carrier to a site in the body; and (c) allowing sufficient time for the expandable polymer to transition from the initial compressed configuration to an expanded configuration, thereby delivering the therapeutic agent. This method may be used to deliver a therapeutic drug anywhere in the body, including lumens, cavities, and solid tissue.

Finally, the invention provides a method of retrieving a matter from a body cavity of a patient using a device with an expandable sleeve. The device comprises (a) a retrieval element adapted for positioning inside the blood vessel, wherein the retrieval element has a proximal and a distal end, wherein the distal end comprises an expandable sleeve and wherein the retrieval element has a channel that extends through the entire length of the retrieval element and the balloon-expandable sleeve, the retrieval element further comprising an inflatable balloon positioned concentrically inside the channel, wherein the balloon, when inflated, is capable of radially expanding the expandable sleeve; (b) an elongated carrier slidably positioned within the channel of the retrieval element, the elongated carrier having a distal portion; and (c) a radially expandable polymer circumferentially attached to the distal portion of the carrier, wherein the expandable polymer has an initial compressed configuration.

The method comprises (a) providing the device with the retrieval element; (b) positioning the retrieval element inside the body cavity; (c) advancing the distal portion of the carrier through the channel of the retrieval element and the expandable sleeve into the body cavity; (d) moving the distal portion of the carrier through or around the matter; (e) allowing sufficient time for the expandable polymer to transition from the initial compressed configuration to an expanded configuration thereby trapping the matter; (f) inflating the balloon, thereby expanding the sleeve; and (g) retrieving, at least partially, the carrier with the expandable polymer in its expanded configuration into the expanded sleeve.

The above-described devices and methods of retrieval of a matter and delivery of a therapeutic agent provide a number of unexpected advantages over the existing devices and methods. The advantages include, but are not limited to, the simple and economical, yet reliable, operation of the devices, which improves the positive outcome of matter removal procedures. The use of a retrieval element according to one of the embodiments of the present invention further ensures safe retrieval of the matter from a body cavity.

Advantageously, the devices of the present invention accommodate attachment of optional steerable flexible tips that simplify navigation of the devices through body cavities such as the vasculature even to sites that are most remote from the entry point of the device. Also, expandable polymers (and foams in particular) used in the present invention allow more effective capturing of matter because of their better surface properties as compared to conventionally used metallic capture devices.

The invention is defined in its fullest scope in the appended claims.

### DESCRIPTION OF THE FIGURES

The above-mentioned and other features of this invention and the manner of obtaining them will become more apparent, and will be best understood by reference to the following descriptions, taken in conjunction with the accompanying drawings, in which:

FIGS. 1A-1C schematically show several embodiments of the device of the present invention;

FIGS. 2A-2E schematically illustrate how the device shown in FIGS. 1A-1C may be used for removing a matter from a body cavity such as a lumen of a blood vessel;

FIGS. 3A-3H schematically show devices in accordance with other embodiments of the present invention; FIG. 31 shows forming a foamlike material from an expandable polymer in accordance with one embodiment of the present invention;

FIGS. 4A-4B schematically show devices in accordance with other embodiments of the present invention; FIGS. 4C-4E schematically illustrate how such devices may be used for removing a matter from a lumen of a blood vessel;

FIGS. 5A-5F show flexibility imparting features added to the expandable polymer in accordance with one embodiment of the present invention;

FIGS. 6A-6B depict an optional retrieval element with a self-deploying sleeve that may be used with devices of the present invention; FIGS. 6C-6E schematically illustrate how such device with the optional retrieval element may be used for removing a matter from a lumen of a blood vessel; FIGS. 6F-6G depict optional retrieval elements in accordance with other embodiments of the present invention;

FIGS. 7A-7F show an optional balloon-expandable retrieval element and its use for removing a matter from a blood vessel in accordance with an embodiment of the present invention;

FIGS. 8A-8B show devices of the present invention having a wire coil running through the expandable polymer in accordance with another embodiment of the present invention; and

FIG. 9 shows delivery of a therapeutic agent into a solid tissue in accordance with one embodiment of the present invention.

### DETAILED DESCRIPTION OF THE INVENTION

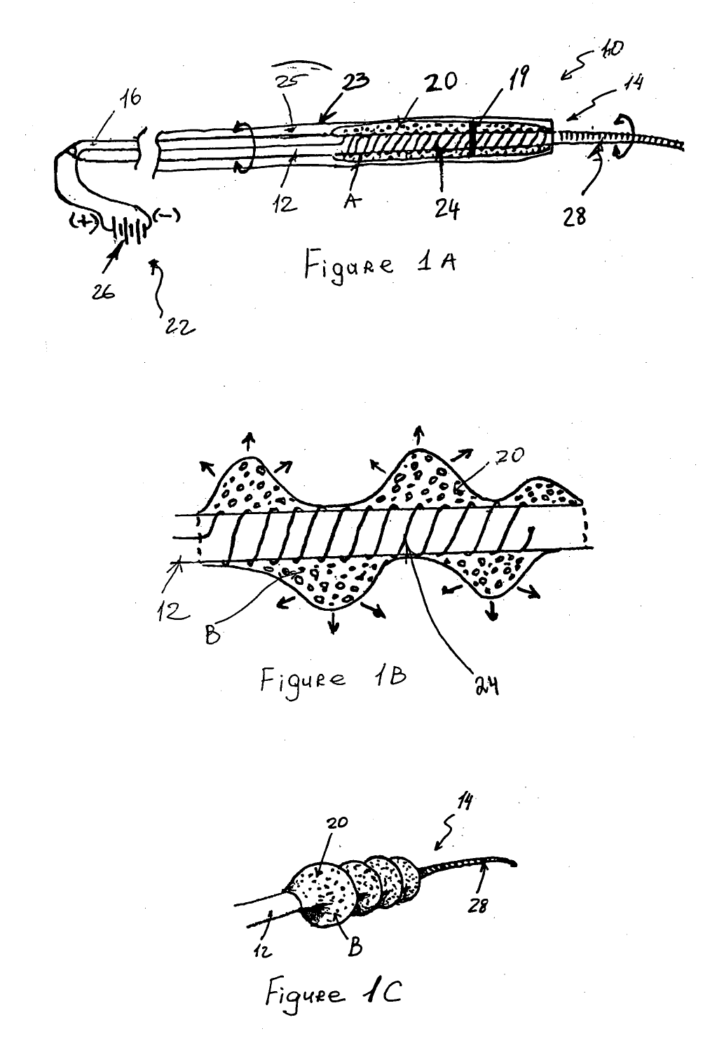

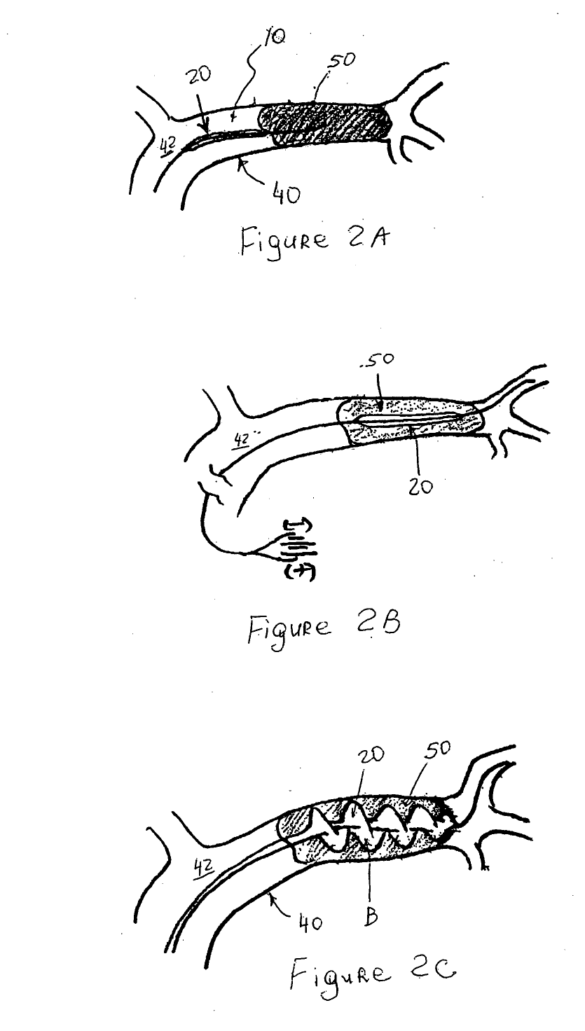

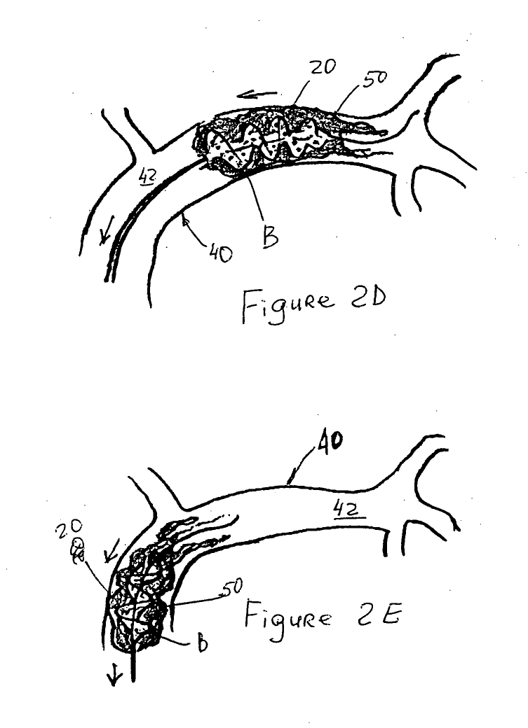

Referring to FIGS. 1 and 2, in one aspect, the present invention is directed to a device 10 for removing a matter from a body cavity of a patient. The patient may be a human or an animal. The device 10 comprises an elongated carrier 12 having a distal portion 14 adapted to move through or within a body cavity of a patient, such as a lumen 42 of a blood vessel 40 and a proximate portion 16. A radially expandable polymer 20 is circumferentially attached to the distal portion 14 of the carrier 12 and adapted to enter a matter 50 blocking the lumen 42 while in a compressed configuration A shown in FIGS. 1A, 2A, and 2B.

It is to be understood that although FIG. 2 shows the device of the present invention being used to remove a matter from a blood vessel, the devices and methods of the present invention may be used in any conduit or cavity inside a patient's body that is naturally existing or surgically made. Examples of such conduits and cavities include, but are not limited to, parts of the alimentary tract, including esophagus, stomach, small and large bowels, anus and rectum; parts of the genitourinary system including renal pelvis, ureter, urethra, spermatic cord, fallopian tubes; the ventricles and cisterns of the brain; the urinary bladder; cysts, vagina; uterine cavity; pseudocysts; abcesses; and fistulae. It also is to be understood that the form of the device depicted in FIGS. 1 and 2 has been chosen only for the purpose of describing a particular embodiment and function of the invention.

The device of the present invention may be used to remove any type of matter, including, but not limited to, clots, emboli, calculi, pieces of atherosclerotic plaque and debris, loose pieces of tissue and neoplasia, thick secretions or fluids, and foreign bodies. The expandable polymer of the device engages the matter from within and drags it from its location into a larger retrieval/guiding catheter located within a body cavity. For example, in one embodiment of the present invention, the device is used to engage a clot in a blood vessel and drag it into a larger retrieval/guiding catheter located in the cervical internal carotid or vertebral artery.

The expandable polymer 20 is capable of transitioning to an expanded configuration B, which is shown, for example, in FIGS. 1B, 2C-2E, and 3A-3G, while inside the matter 50 to penetrate and engage it from within. The polymer may be attached to the carrier using any method of attachment that provides a reliable immobilization of the polymer on the carrier. Such methods are well known and include, but are not limited to, the use of a biocompatible epoxy adhesive, welding of a metal wire element running through the expandable polymer to the carrier, and/or trapping a collection of expandable polymer mechanically between two widened zones on the carrier. In one embodiment, the expandable polymer exhibits an adhesive property to the matter.

In one embodiment, the polymer is a shape-memory polymer selected from a group consisting of polyurethane, polyethylene, polyethylene terephthalate, polyisoprene, styrene-butadiene copolymers, copolyester, ethylene-vinylacetate and other ethylene copolymers, acrylates including, but not limited to polyacrylamide gel and polyacrylic acid, norbornane, polynorbornene, and polystyrenes. Using a shape memory polymer in the device of the present invention allows the device to pass into the body cavity and navigate into the vicinity of a matter to be removed in a compressed configuration, which decreases the possibility of damaging the walls of the body cavity. For example, the device may be easily passed through a lumen of an intracranial microcatheter and subsequently be navigated through or into the vicinity of a matter blocking a blood vessel without damaging the walls of the blood vessel.

The polymer may contain a predetermined amount of a therapeutic agent. In one embodiment, the optional therapeutic agent is released when the polymer 20 transitions from the compressed configuration A to the expanded configuration B. For the purposes of the present invention, the phrase “therapeutic agent is released when the polymer 20 transitions” refers to a release of the therapeutic agent during or after the transition between the compressed configuration A and the expanded configuration B. One of the advantages of using the device of the present invention having a radially expandable polymer for drug delivery is a localized targeting of pathology and the avoidance of systemic delivery and undesirable systemic effects of a drug or vector.

The therapeutic agent is not limited to a particular chemical or biological group. Suitable therapeutic agents are well known to physicians and are based on a patient's state of a disease. Some appropriate therapeutic agents include, but are not limited to, an anti-thrombogenic, thrombolytic, anti-proliferative, anti-spasmodic, anti-coagulant, anti-platelet adhesion drugs, endothelial cells, and gene vectors. In one embodiment, the thrombolytic drug is selected from a group consisting of tissue plasminogen activator (t-PA), streptokinase, a calcium ion influx inhibitor, urokinase, and their analogs.

The transition of the polymer between the compressed configuration A and the expanded configuration B may be triggered by a physiological stimulus, by an external stimulus, by a mechanical device or force, or by their combinations. Examples of the physiological stimulus include, but are not limited to, body temperature, blood pH, an ion concentration in blood, and overall blood composition. Examples of the external stimulus include, but are not limited to, solutions, the introduction of which into the blood vessel causes changes in the local chemical environment, external temperature, light, magnetic field, ultrasound, radiation, and electrical field.

Examples of mechanical devices and forces include, but are not limited to, various types of sheaths, casings, covers, and other types of restrainers that are capable of retaining the expandable polymer in the compressed configuration. Removal of such restrainers leads to transition of the polymer into the expanded configuration. Referring to FIG. 1, in one embodiment, the device 10 of the present invention further comprises a delivery device 23 adapted for positioning inside the cavity and having an internal lumen 25, wherein the distal portion 14 of the carrier is slidably positioned within the lumen 25, wherein the polymer remains in the compressed configuration inside the delivery device and the polymer transitions into the expanded configuration when it exits the delivery device or delivery device is removed.

In one embodiment, the polymer is a hydrogel. A hydrogel is a three-dimensional network of hydrophilic polymer chains and water that fills the space between polymer chains. Typically, hydrogels are two- or multicomponent systems, in which polymer chains are cross-linked through either chemical or physical bonding. In physical gels (pseudogels), the chains are connected by electrostatic forces, hydrogen bonds, hydrophobic interactions, or chain entanglements. In chemical hydrogels, chains are linked by covalent bonds. Because of the hydrophilic nature of polymer chains, hydrogels absorb water and swell in the presence of abundant water. Typically, water constitutes at least 10% of the total weight (or volume) of a hydrogel.

Any hydrogels may be used for the purposes of the present invention as long as they are capable of transitioning from a compressed into an expanded configuration in a controllable fashion. Examples of hydrogels include, but are not limited to, polyethylene oxide, polyvinyl alcohol, polyvinylpyrrolidone, polyhydroxyethyl methacrylate, polyetherpolycarbonatecollagen, and polysaccharides.

The transition of the hydrogel of the present invention into the expanded configuration may be triggered by a number of internal and external stimuli, including, but not limited to, changes in hydration, pH, solute concentration (e.g., glucose concentration), the ionic environment (including calcium, magnesium, potassium, and sodium), local light levels, temperature, electric field, magnetic field, radiation, and ultrasound. For example, in one embodiment, a hydrogel that swells at a predetermined time as a result of the absorption of blood from the blood vessel is used.

In another embodiment, a biocompatible triggering fluid is applied to a hydrogel to initiate the transition from the compressed to the expanded configuration. Triggering solutions are well known in the art and may include fluids having a predetermined pH or composition that cause the hydrogel to swell and to transition into the expanded configuration. For example, lactated ringers solution, glucose, or saline may be used.

In another embodiment, the polymer is a shape-memory foam. Shape-memory polymer foams are materials that can be formed into a desired shape (“expanded configuration”) and then can be constrained into a deformed configuration (“compressed configuration”) at a temperature higher than the glass transition temperature point (Tg) of the polymer and then kept compressed at a temperature lower than the Tg. The original configuration of the foam can be at least partially recovered when the foam is again heated to and maintained at a temperature higher than the Tg.

Any shape-memory foam may be used for the purposes of the present invention as long as it is capable of transitioning from a compressed into an expanded configuration in a controllable fashion. Examples of such foams include, but are not limited to, polyurethane, a cross-linked ethylene-vinyl acetate, and polyethylene copolymers. Formulations and properties of shape-memory foams are well known to those skilled in the art and are described, for example, in the following references, each of which is incorporated herein by reference: U.S. Pat. Nos. 5,049,591; 6,702,976; 5,032,622; 5,145,935; 5,188,792; 5,242,634; 5,418,261; 6,102,933; 6,156,842; 6,583,194; U.S. Pat. Appl. Publ. No. U.S. 2002/0101008 A1; Metcalfe et al., “Cold hibernated elastic memory foams for endovascular interventions;” Watt et al., “Thermomechanical properties of a shape memory polymer foam.” In one embodiment, a shape-memory foam is a polyurethane foam. Such foams can be formulated to provide a desired Tg and cell size. In one embodiment, the foam's cell size is chosen to maximize its adhesiveness to the matter.

Although a variety of glass transition temperatures may be chosen, in one embodiment, Tg is below a body temperature (i.e., <37-38° C.) and the foam spontaneously transitions into the expanded configuration after being exposed to the body temperature for a predetermined time. In another embodiment, Tg is above a body temperature (i.e., >37-40° C.). In this embodiment, the device further comprises a source of heat 22. Those skilled in the art would recognize that a wide range of heat sources, including, but not limited to, electrical resistance, inductive, optical, and convective heating elements, may be used.

In one embodiment, the source of heat is an electrical resistance element comprising a metal or a semiconductive plastic coil 24 that is circumferentially attached to the distal portion 16 of the carrier 12 and electrically connected to a controller 26, which is located outside of the patient, through an insulated pathway. The controller 26 delivers direct electrical current at the appropriate voltage to the resistive heater to heat the foam layer to its Tg, thus enabling the foam to expand fully to its expanded configuration.

The controller is capable of adjusting a voltage applied to the coil 24 to maintain a predetermined temperature. The controller works by measuring the resistance within the circuit. This provides an indirect, but reliable, measurement of the resistive heater's temperature, since, as the heater's temperature rises, so does the circuit's resistance, in a predictable manner. Thus, as the circuit's resistance rises above an undesirable level, the controller shuts off current flowing to the heater. The controller will continue to assess the circuit's resistance by short bursts of current until the resistance falls to just below the critical level, at which point, direct current will again be delivered to the resistive heater at an appropriate voltage. The current flow continues until the critical resistance level is again exceeded, again terminating continuous current flow. This continuous feedback mechanism used by the heater controller maintains the heater's wire coil within a narrow temperature range around the foam's Tg.

Optionally, the heater controller also may include a timer that allows activation of the coil for an appropriate length of time, which is sufficient to ensure full expansion of the compacted foam segment. The heater's wire coil may be made of any metal or semiconductive plastic. In one embodiment, tungsten is used.

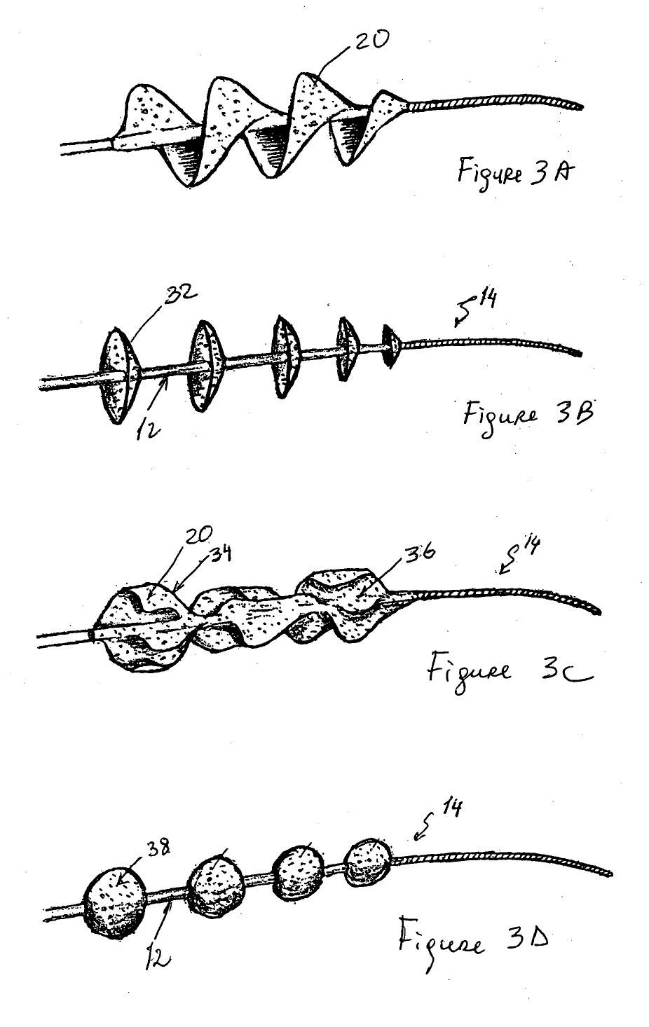

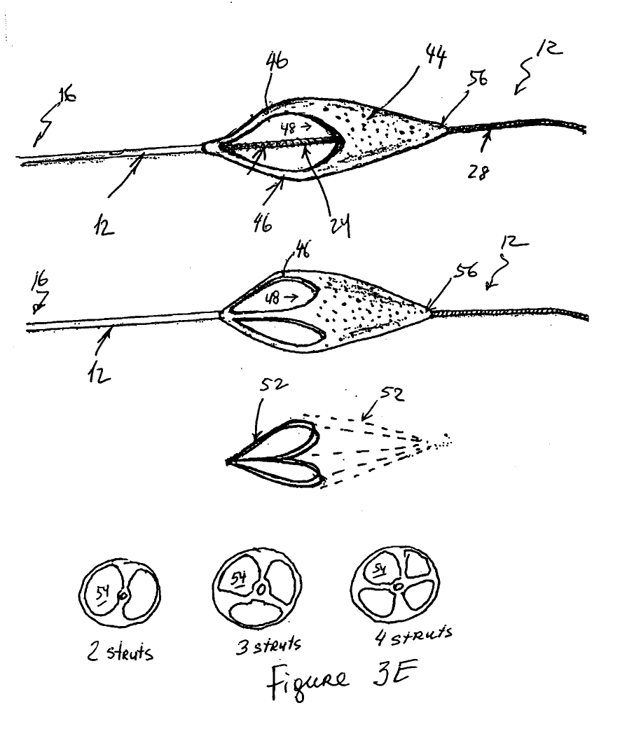

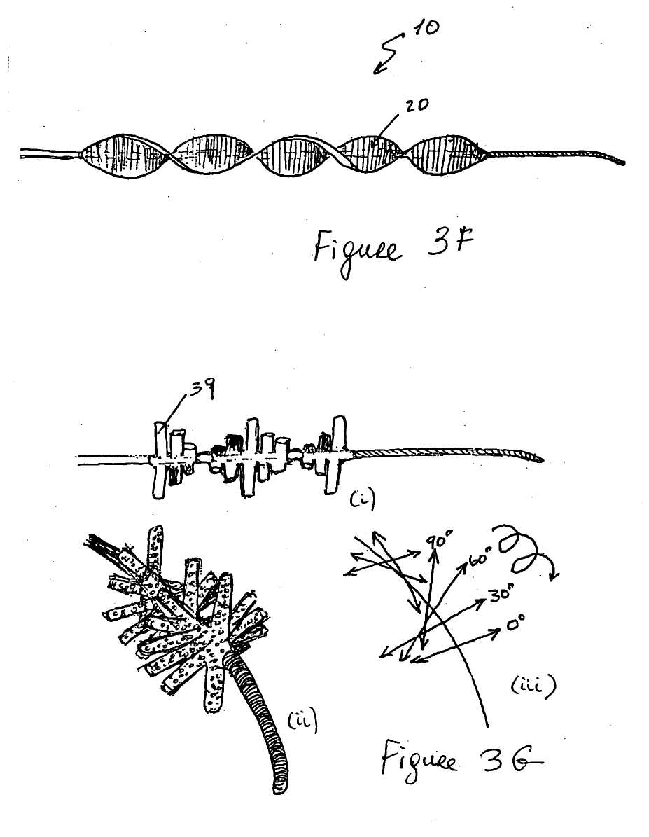

The expandable polymer may be a material other than foam or hydrogel as long as it can be forced into a compressed configuration and is capable of transitioning into an expanded configuration. As shown in FIG. 3I, in one embodiment, cells, holes, and/or cavities 7 are machined by using a laser beam 9, a mechanical tool, or other means in a solid polymer 5 to impart foamlike texture and shape-memory properties. Expandable polymers of the present invention may have a reticular pattern to increase their surface area for contact with the matter.

In one embodiment, the distal portion 14 of the elongated carrier 12 further comprises a steerable tip 28 (FIGS. 1A and 1C). The steerable tip may be a shapable platinum or stainless steel wire. The optional steerable flexible tip of the present invention advantageously simplifies navigation of the device through the body cavity, such as vasculature, even to sites that are most remote from the entry point of the device.

The device 10 of the present invention also may serve to deliver catheters and other devices mounted on catheters in much the same way as an exchange guidewire. Examples of such devices may include angioplasty balloons, stents, or microcatheters. This may be a particularly useful feature if, during attempted removal of an obstructing clot during treatment of acute stroke, a narrowing or stenosis in a blood vessel is discovered. When a stenosis is discovered while retrieving a clot and retracting a conventional Concentric Retriever device having a “cork screw” configuration, the coil straightens out and the grip on the clot is lost. In addition, in some situations, the coil of the Concentric device could break off and/or injure the blood vessel as attempts to drag it across a stenosis are made.

To ensure that matter being removed from the body cavity (e.g., a clot being removed from a blood vessel) is not lost when the device 10 is retrieved through a narrowed area, an angioplasty balloon, stent, or another similar device may be advanced over the proximal end of the carrier 12 and delivered to the site of the stenosis (downstream of the expandable polymer). The device may then be used to expand the narrowing and to enable removal of the clot retrieval device along with the matter. In one embodiment, the procedure described above may be used to perform an angioplasty to improve the luminal diameter of a narrowed blood vessel. The device 10 of the present invention also may serve as a protective filter in a blood vessel, distal to a site of angioplasty and/or stent placement, especially at intracranial sites, and have sufficient length to serve the function of an exchange wire while delivering angioplasty balloon catheters and stents to the treatment site.

The elongated carrier 12 of the present invention may be a guidewire or a catheter. For example, in one embodiment, a steerable guidewire with a preferred diameter range of 0.008″-0.018″, but possibly up to 0.038″ is used. The guidewire may be constructed of one or more fiber optic fibers, capable of transmitting light to the distal end of the device. In one embodiment, the light consists of laser light of one or more different wavelengths and is capable of effecting a change in the expandable polymer configuration in one or more locations.

The polymer in its expanded configuration may have any shape and form as long as it allows penetration and engagement of the matter to be removed from within. For example, referring to FIGS. 3A-3G, it may be in a form of a coil or a screw-like structure (FIG. 3A), a twisted ribbon (FIG. 3F), a formation of one or more disks 32 (FIG. 3B), a parachutelike structure (FIG. 3E), a formation comprising a plurality of ridges 34 and troughs 36 (FIG. 3C), a formation of one or more spheres or globes 38 (FIG. 3D), and a formation comprising a plurality of outwardly extending spears 39 (FIG. 3G).

In several embodiments shown in FIGS. 3B, 3D, and 3G, the device comprises an elongated carrier 12 having a distal portion 14 adapted to move through the lumen and at least two isolated formations (e.g., 32 or 38) of radially expandable polymer attached to the distal portion of the carrier. Each formation encloses the entire circumference of the carrier. The formations are adapted to move through or around the matter while having a compressed configuration and capable of transitioning to an expanded configuration to trap the matter therebetween. In one embodiment, the polymer is a hydrogel or a foam.

The device may comprise a plurality of progressively decreasing in size formations of the radially expandable polymer. Such configuration advantageously permits the retrieval of clots, emboli, or foreign bodies from both larger and distally smaller vessels with the same device. The progressively decreasing in size formations may be disks (FIG. 3B), spheres (FIG. 3D), outwardly extending spears (FIG. 3G), or configurations comprising a plurality of ridges and troughs.

In one embodiment shown in FIG. 3E, the expanded configuration has a parachutelike structure surrounding and attached to the carrier 12. The parachute-like structure comprises a basket portion 44 for collecting the matter and at least two supporting struts 46, preferably, 2-6 supporting struts. The basket portion 44 has a hollow interior 54 and an opening 48 facing the proximate portion 16 of the carrier 12. The closed bottom 56 of the basket portion 44 is adjacent to the distal portion 14 of the carrier 12. The distal portion may optionally comprise a steerable shapable tip 28. Also, optionally, the device may have an external source of heat with an electrical resistance element comprising a metal or a semiconductive plastic coil 24.

Optionally, the struts may be reinforced by embedded wire loops or an embedded polymer fiber network 52 that would extend through the struts and into the distal cone portion of the parachute. In one embodiment, the wire loops are made of a shape-memory material such as nitinol. In another embodiment, the polymer fiber network is made of fibers selected from a group consisting of polyamide (or polyaramide) fibers such as those sold under the trademark KEVLAR® (DuPont, Richmond, Va.), polyethylene fibers, and liquid crystal polymer fibers, such as those sold under the trademark VECTRA® (Celanese, Germany). Preferably, the basket portion is positioned distal to the matter that needs to be removed and, then is gently withdrawn to retrieve the matter. In one embodiment, the struts aid in the retrieval of the basket portion by allowing it to be collapsed and forced down into a retrieval catheter (not shown).

In one embodiment shown in FIG. 3G, the polymer in its expanded configuration may comprise a formation of a plurality of outwardly extending spears 39. The spears may have a spiral configuration, as demonstrated in FIGS. 3G(ii) and (iii).

The polymer 20 may be capable of storing torque energy when in compressed configuration and releasing it in the expanded configuration, in much the same way that a twisted rubber band provides a transient surge of energy to a model airplane (i.e., the potential energy stored in the wound-up rubber band powers the plane's propeller). Accordingly, in one embodiment shown in FIG. 3H, a band of expandable polymer 20 (FIG. 3H(i)) is woundup (FIG. 3H(ii)) and unwinding of the band causes torque to drive a microdevice 37, optionally attached at the distal end 14 of the carrier, for a predetermined time (FIGS. 3H(iii)-3H(iv)). The microdevice may be a tiny propeller, a screw, an auger, or other small device. In one embodiment, the microdevice is capable of dissolving or fragmenting a clot or atheromatous plaque or debris. In another embodiment, the microdevice assists in retrieval of the matter.

Optionally, the polymer 20 may be a temperature sensitive foam or a polymer fiber band. The band may be wound up at a temperature above Tg and then cooled down below Tg to stabilize the polymer in the twisted configuration (FIG. 3H(iii)) and to store its potential energy in a stable form. When the twisted band is placed into the environment with a temperature above Tg, the polymer is activated and releases the torque stored in the twisted band. In one embodiment, the device has an external source of heat, such as the resistive heater 22 described above, for activating the foam. Optionally, the polymer may be a foam reinforced by fibrous resilient material.

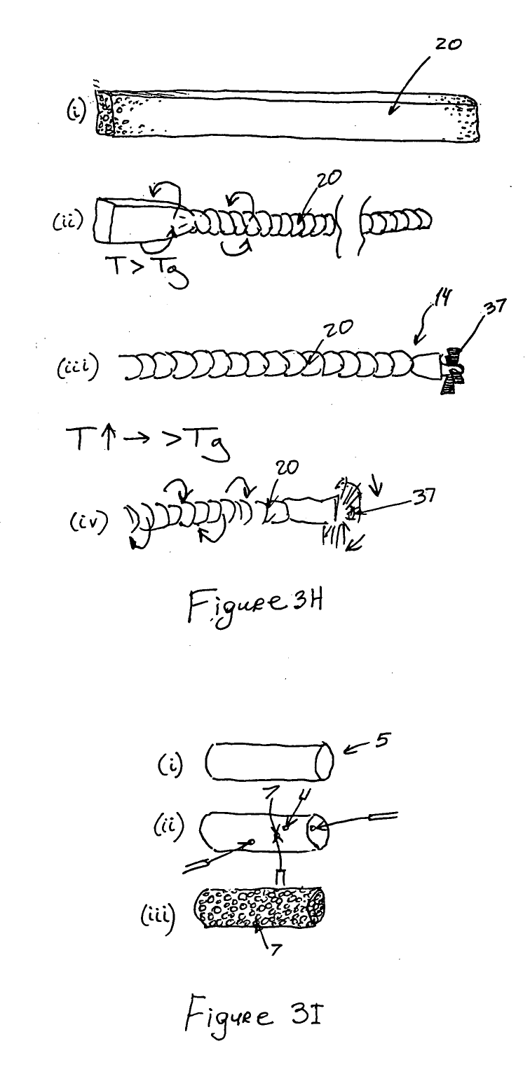

Referring to FIG. 4A, the device of the present invention also may include a thin polymer coating 60 applied to the radially expandable polymer. The coating may be used to prevent fragmentation of the expandable polymer. The coating also may be used to impart desirable physical and chemical properties. For example, in one embodiment, the coating has hydrophilic and/or lubricious properties to aid in advancement of the device inside or through the body cavity. In another embodiment, the coating is used to provide a magnetic field, a positive charge, a negative charge, or their combination to the expandable polymer. In one embodiment, other portions of the device are coated to provide a desirable physical or chemical property.

For example, a magnetically or electrically charged surface of the device may advantageously allow the attraction or repellent of matter inside the body cavity. Alternatively, the expandable polymer itself may provide a desirable surface charge, magnetic field, or other desirable physical or chemical properties. The charge or magnetic field may be an intrinsic property of the polymer, produced by chemical modification of the polymer's surface, or induced by application of an external energy or a source of magnetism. In one embodiment, the charge is induced by an external electrical source or a thermocouple located inside the device. In another embodiment, a magnetic field is created by a fixed permanent magnet or an electromagnet located in the distal portion 14 of the device 10. The electromagnet may be induced by an electric current applied through wires running through the device, as seen in FIG. 1A. Depending on the amount of current applied, the configuration of the coils, and the resistive nature of the wire, any combination of resistive heat generation and magnetic field generation may be accomplished.

In one embodiment, the coating is made of a semipermeable elastomeric material such as latex, PVC, silicone rubber, and silicone-modified styrenic thermoplastic elastomers sold under the trademark C-FLEX® (Consolidated Polymer Technologies, Inc., Clearwater, Fla.). The coating may be in a form of a sleeve running the length of the device. When a hydrogel is used, the sleeve may advantageously provide a means of injecting a triggering fluid for initiating expansion of the hydrogel. Optionally, the expandable polymer or the optional coating may contain a medical composition that prevents thrombus formation on the expandable polymer. In one embodiment, the medical composition comprises heparin and/or an anti-platelet adhesion agent to help prevent thrombus formation.

Referring to FIG. 1A, the device may further include radiopaque markers 19 (such as platinum) or a material (such as barium sulfate) that will allow the operator to determine fluoroscopically the location of the device. Also, radiopaque markers may be incorporated into the expandable polymer to allow the operator to see whether the expandable polymer is in a compressed or expanded configuration.

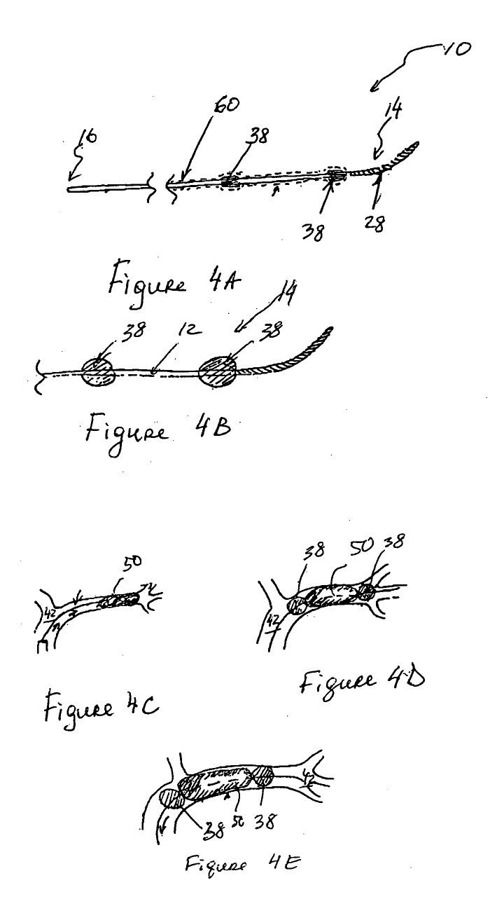

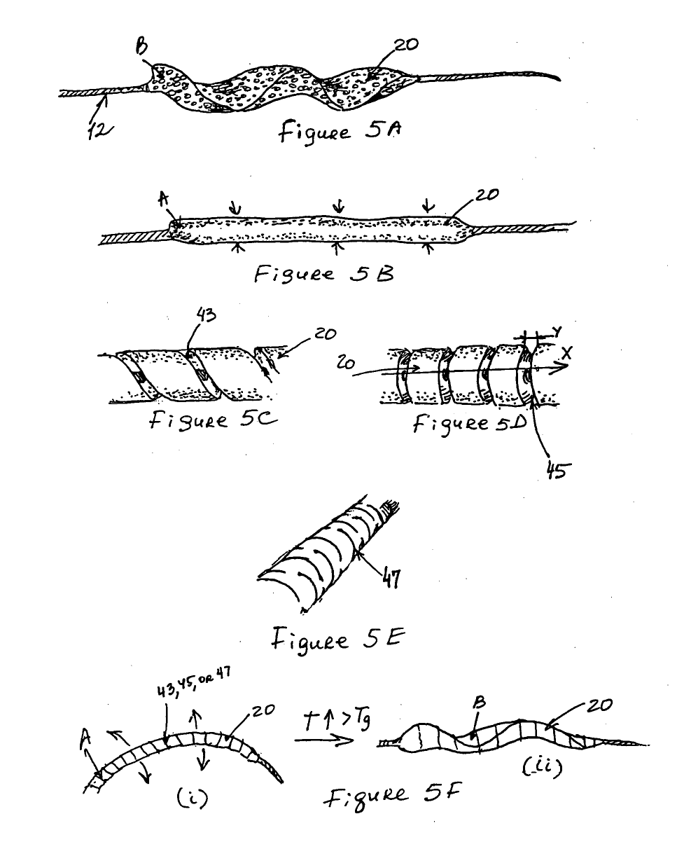

When in the compressed configuration, the expandable polymer may have a reduced flexibility, which may negatively affect maneuverability of the device. Referring to FIGS. 5A-5F, the compressed polymer may be etched or machined to create at least one feature imparting a desired level of flexibility to the carrier with the polymer in the compressed configuration. For example, the feature may be a cut, groove, slot, or indent. In one embodiment, a desirable shape A of expandable polymer 20 is created and attached to the carrier 12. Then the expandable polymer 20 is heated above Tg and compressed to form a compressed configuration A (FIG. 5B). The expandable polymer 20 retains its compressed configuration until it is exposed to a temperature above Tg.

To improve flexibility and maneuverability of the expandable polymer, cuts, grooves, slots, or other features are created using a laser beam, a mechanical blade, or other suitable tool. In one embodiment, a continuous spiral cut 43 is formed along the length of the expandable polymer (FIG. 5C). In another embodiment, a plurality of cuts or slots 45 are formed perpendicularly to a long axis X of the carrier, with each slot or cut being offset circumferentially by a distance Y from an immediately preceding slot or cut. As shown in FIG. 5E, in another embodiment, repeating orthogonal cuts 47 may be made to create a complex multiple cut pattern. These and other features afford a greater flexibility to the compressed polymer (FIG. 5F(i)). Yet, when expanded, the expandable polymer substantially returns to its pre-cut expanded configuration B (FIG. 5F(ii)).

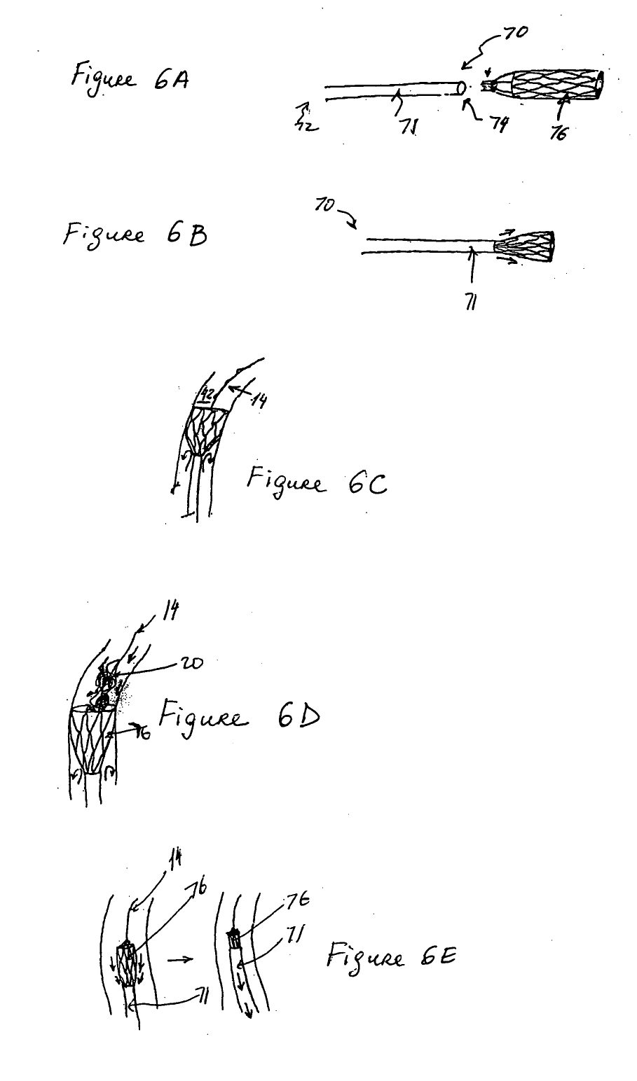

When the obstructing matter is captured by the device of the present invention, it is highly desirable to remove it from the body in a manner that would minimize the risk of its fragmentation or loss. In one embodiment illustrated in FIGS. 6A-6E, this risk is mitigated by using a retrieval element 70 adapted for positioning inside a body cavity, such as a lumen 42 of a blood vessel. The retrieval element may comprise a guiding catheter 71 with a proximal end 72 and a distal end 74. The distal end 74 comprises a self-deploying expandable sleeve 76. The retrieval element has a channel that extends through an entire length of the guiding catheter 71 and the expandable sleeve 76. The distal portion 14 of the carrier 12 is slidably positioned within and adapted to move through the channel into the body cavity. Preferably, the expandable polymer 20 in its expanded configuration is capable of being at least partially retrieved into the expandable sleeve, 76 as shown in FIG. 6D.

Optionally, as shown in FIG. 6E, the sleeve is capable of packaging the entire radially expandable polymer in its expanded configuration inside the sleeve. In one embodiment shown in FIGS. 6C-6E, sleeve, 76 in its expanded form, advantageously blocks antegrade blood flow and creates retrograde blood flow toward the open sleeve.

Any construction of the sleeve 76 is acceptable, as long as it is self-deploying and expandable. In one embodiment shown in FIGS. 6A-6E, the sleeve comprises a wire core in a form of a plurality of wire ring components forming a netlike configuration. Such multiple wire ring components may be welded together at several points to provide some flexibility of the design. Very thin (0.004″-0.008″ diameter) wire may be used. The wire may be made of a metal such as titanium or an alloy, such as nitinol, ELGILOY®, Ni/Co/Cr/Mo/Fe alloy (Elgiloy Limited Partnership), and steel. Optionally, a thin cylindrical polyurethane or PTFE sleeve may be attached to the wire core by adhesive application, small sutures, “sandwiching” the wire rings between two thin polymer layers, or some other suitable method.

Preferably, as shown in FIG. 6E, the expandable sleeve may be contained in its collapsed configuration within the distal end 74 of the guiding catheter 71 (e.g., 8-9 F guiding catheter) used for the introduction of the device 10 of the present invention into the lumen of the blood vessel. Once the device 10 is withdrawn into the open sleeve 76 with its captured material (FIG. 6D), the sleeve is withdrawn back into the guiding catheter 71 (FIG. 6E), thus securely packaging the device 10 and the captured matter to allow their safe retrieval from the body.

Referring to FIGS. 6F-6G, alternative designs are possible for the expandable sleeve. For example, as shown in FIG. 6F, the sleeve may comprise a plurality of right-angle loops 81 attached to a pusher/retraction wire 82 and having an attached conelike polymer sleeve 84. In another embodiment shown in FIG. 6G, multiple rings 86 made of a shape-memory material and having progressively enlarging diameters are joined at opposite ends a and b. A cone-like polymer sleeve 84 is attached to the rings. Both of these designs may be contained in a collapsed state within the distal length of the guiding catheter and would be deployed by pushing them out of the end of this catheter. After the device 10 with the captured matter is pulled back into the sleeve 76, the sleeve is collapsed by pulling it back into the catheter, thus allowing safe retrieval of the captured material.

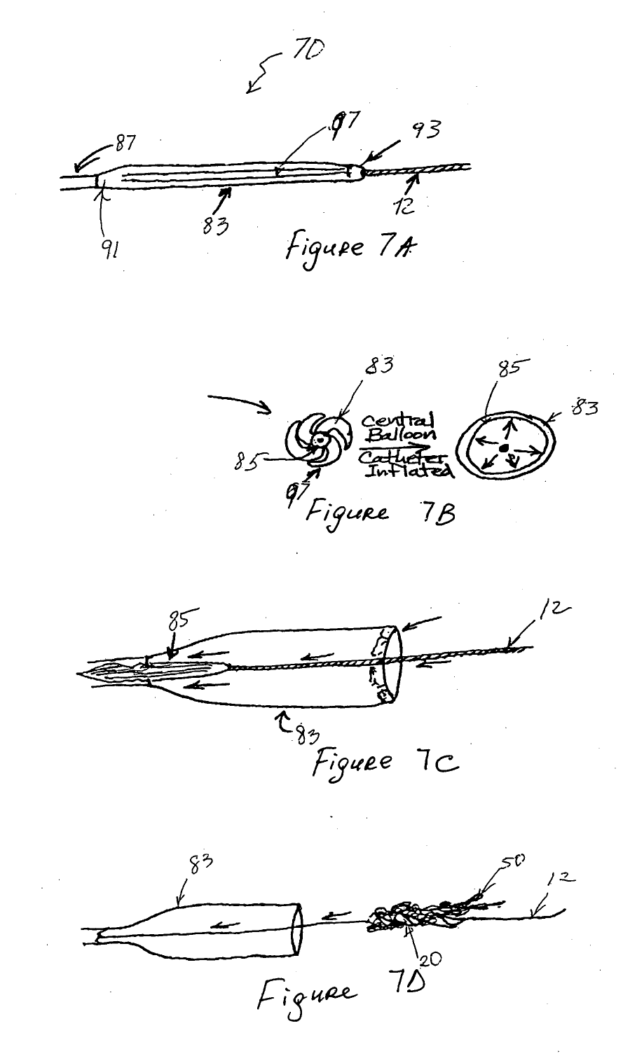

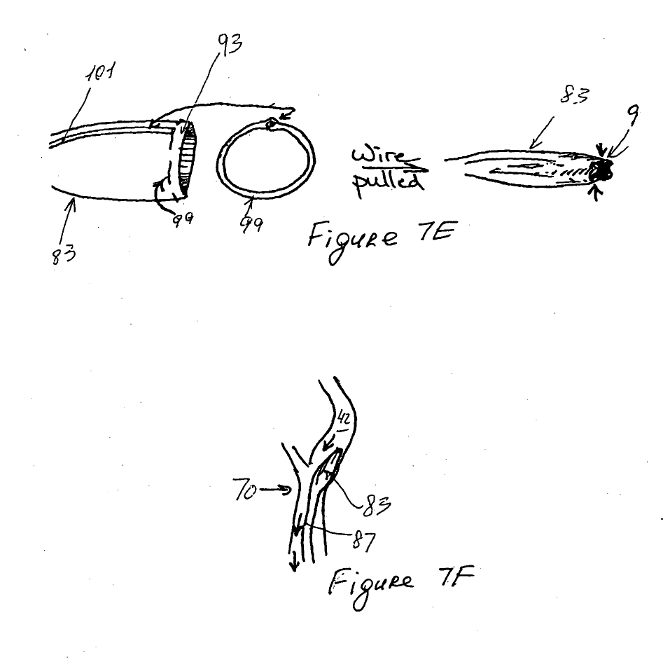

Referring to FIGS. 7A-7F, retrieval element 70 may have an inflatable removable balloonlike structure (referred to as balloon) 85 for expanding the sleeve 83. In this embodiment, the expandable sleeve has a proximal end 91 and a distal end 93. The proximal end may be attached to a nonexpandable shaft 87. In one embodiment, the shaft is a 7-8 F shaft. The distal end 93 may be tapered. Preferably, at least the distal end of the expandable sleeve is made of an elastomeric material such as SILASTIC® (Dow Corning, Midland, Mich.) or C-FLEX® (Consolidated Polymer Technologies, Inc., Clearwater, Fla.) material. The balloon 85 is positioned concentrically inside the expandable sleeve 83. In one embodiment, the elongated carrier 12, such as 0.035″-0.038″ guidewire, is slidably positioned through the center of the balloon 85. As shown in FIG. 7B, the expandable sleeve 83 may be folded to form folds or “wings” 97 and wrapped tightly like an angioplasty balloon. When the balloon 85 is inflated, it expands the sleeve 83 from within. Then, as shown in FIG. 7C, the balloon 85 may be deflated and removed through the nonexpandable shaft 87. Referring to FIG. 7D, the expanded sleeve 83 accommodates, at least partially, polymer 20 in its expanded configuration with the captured matter 50. Referring to FIG. 7F, the retrieval element 70 with the trapped matter may then be removed from the body cavity 42.

Referring to FIG. 7E, the distal end 93 of the sleeve 83 may be optionally contracted after the expandable polymer with the captured matter is retrieved into the sleeve. In one embodiment, a loop structure 99 is placed circumferentially at a distal end 93 of the expandable sleeve 83. Another longitudinal structure 101 is placed longitudinally through a separate lumen in the sleeve 83 and is connected to the loop structure 99. The loop structure and the longitudinal structure may be made of any flexible material such as a metal wire, purse string, or radiopaque suture. When the longitudinal structure 101 is retracted, the distal end 93 of the sleeve 83 contracts and captures the trapped matter 50.

Referring to FIGS. 1-4, in still another aspect, the present invention provides another device for retrieving a matter 50 from a body cavity such as a lumen 42 of a blood vessel. The device comprises an elongated carrier 12 having a distal portion 14 adapted to move through the lumen and a radially expandable polymer 20 circumferentially attached to the distal portion 14 of the carrier. The polymer is adapted to move through the matter while having a compressed configuration and capable of transitioning to an expanded configuration to engage the matter for retrieval from the lumen. In this embodiment of the invention, the transition of the polymer is triggered by a physiological stimulus.

The present invention further provides a method of retrieving a matter from a lumen of a blood vessel. The method comprises (a) providing a device with a retrieval element having a balloon-expandable sleeve that was described above; (b) positioning the retrieval element inside the body cavity; (c) advancing the distal portion of the carrier through the channel of the retrieval element and the balloon-expandable sleeve of the retrieval element into the body cavity; (d) moving the distal portion of the carrier through or around the matter; (e) allowing sufficient time for the expandable polymer to transition from the initial compressed configuration to an expanded configuration, whereby trapping the matter; (f) inflating the balloon, whereby expanding the sleeve; and (g) retrieving, at least partially, the carrier with the expandable polymer in its expanded configuration into the expanded sleeve.

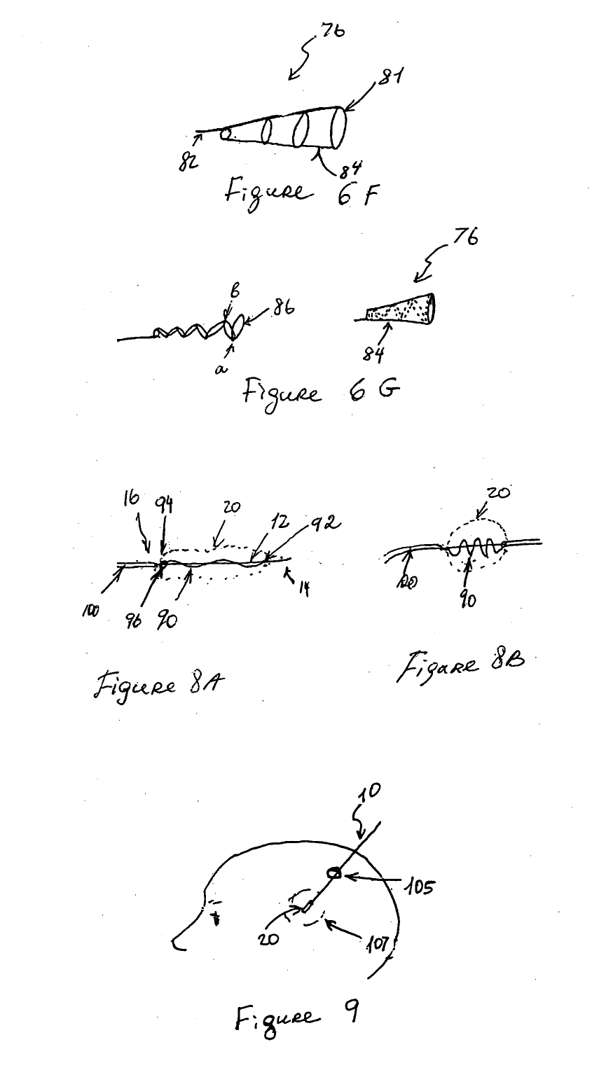

As shown in FIGS. 8A and 8B, the profile of the expandable polymer used to capture the obstructing matter could be significantly enlarged by a wire coil 90 running through the expandable polymer 20 along the carrier 12. The wire coil 90 has a first end 92 fixedly attached by welding or some other means to the distal portion 14 of the carrier and a second end 94 movably attached to the proximate portion 16 of the carrier 12. The proximal end 94 of the coil may have a small loop 96 or a coaxial metal or plastic tube (not shown) keeping the coil attached to the carrier 12, but allowing it to slide freely along the carrier 12.

The device further comprises a barrier circumferentially attached to the carrier, wherein the barrier prevents movement of the second end when the carrier is pulled back, whereby the coil radially expands. For example, the device may have a more proximal microcatheter 100 or hypotube, such that when the central wire is pulled back through this microcatheter or hypotube, the free-sliding proximal connection of the coil would be held stationary, thus foreshortening the coil and increasing its diameter (FIG. 7B).

Referring to FIGS. 2A-2E, the present invention further provides a method of retrieving a matter 50 from a body cavity such as a lumen 42 of the blood vessel. The method comprises (a) providing the device 10 described above; (b) positioning the device inside the lumen 42 of the blood vessel; (c) allowing sufficient time for the expandable polymer 20 to expand and engage the matter 50 from within; and (d) removing the device, whereby removing the matter.

Referring to FIGS. 4A-4D, the present invention further provides a method of retrieving a matter 50 from a body cavity such as a lumen 42 of a blood vessel. The method comprises (a) providing the device 10 described previously and shown in FIG. 4A, the device having at least two isolated formations 38 of radially expandable polymer attached to the distal portion 14 of the carrier 12; (b) positioning the device inside the lumen 42 of the blood vessel; (c) passing at least one formation 38 through or around the matter 50; (d) allowing sufficient time for the isolated formations 38 to expand, whereby trapping the matter between the formations; and (e) removing the device, whereby removing the matter.

Referring to FIGS. 2A-2E and 4A-4D, the present invention further provides a method of retrieving a matter 50 from a body cavity such as a lumen 42 of a blood vessel. The method comprises (a) providing the device 10 described previously; (b) positioning the device inside the lumen 42 of the blood vessel; (c) allowing sufficient time for a physiological stimulus to act on the expandable polymer to cause its transition from the initial compressed configuration to an expanded configuration, whereby engaging the matter in a way that allows its removal; and (d) retrieving the device, thereby removing the matter.

As was discussed in more detail above, the physiological stimulus may be body temperature, blood pH, an ion concentration in blood, and blood composition. The polymer may be a hydrogel or a foam. In one embodiment, the polymer is a hydrogel and the transition of the hydrogel into the expanded configuration is triggered by hydration of the hydrogel inside the blood vessel.

In yet another aspect, the present invention provides a method of localized delivery of a therapeutic agent. The method comprises (a) providing a removable device having a carrier with a distal portion and a radially expandable polymer circumferentially attached to the distal portion of the carrier, wherein the expandable polymer has an initial compressed configuration; (b) advancing the distal portion of the carrier to a site in a body; and (c) allowing sufficient time for the expandable polymer to transition from the initial compressed configuration to an expanded configuration and delivering the therapeutic agent. There is no limitation on a type of the site in the body to which this method could be applied. The site may be a lumen, such a lumen of a blood vessel, a lumen of the alimentary tract, including esophagus, stomach, small and large bowels, anus and rectum, or a lumen of the genitourinary system, including renal pelvis, ureter, urethra, spermatic cord, fallopian tubes, a cavity, such as the ventricles and cisterns of the brain, as well as the urinary bladder, cysts, vagina, uterine cavity, pseudocysts, abcesses, fistulae, surgically created conduits, and cavities or a solid tissue, such as liver, spleen, pancreas, brain, bone, muscle, tumors, testes, ovaries, uterus, lymph nodes.

For example, as shown in FIG. 9, the device 10 of the present invention may be placed in proximity of a brain tumor 107 through a burr hole 107 drilled in the skull of a patient. The device may be placed with the assistance of CT, MR, stereotactic, or other means. The expandable polymer 20 is then activated to release a suitable therapeutic agent. The expandable polymer may be activated by a physiological and/or an externally applied stimulus, as discussed in detail above.

The foregoing is meant to illustrate, but not to limit, the scope of the invention. Indeed, those of ordinary skill in the art can readily envision and produce further embodiments, based on the teachings herein, without undue experimentation.

### EXAMPLE 1

Under fluoroscopic and/or digital roadmap imaging, an appropriate guiding catheter (with or without a distal occlusive balloon) is navigated into the cervical artery (i.e., the internal carotid or vertebral artery) serving the distal intracranial circulation affected by the occlusive thromboembolus or intraluminal foreign body (“target”). Coaxially through this guiding catheter, an appropriate microcatheter (with O.D.=2-3 F and I.D.=0.018″-0.025″) is maneuvered over a steerable microguidewire (approximate diameter=0.014″) under fluoroscopic guidance into the affected intracranial artery just proximal to the target. The aforementioned microguidewire is removed from the microcatheter, and a device with foam circumferentially attached to a wire, in accordance with one embodiment of the present invention, is advanced coaxially through the microcatheter. Using digital roadmap imaging and/or regular fluoroscopy, the device is then navigated through the target.

Next, the heater controller attached to the proximal external end of the device is activated. The resistive heater subjacent to the compressed foam layer raises the temperature of the compressed foam segment to the Tg for several minutes. The foam begins to expand, assuming its expanded configuration as revealed by radiopaque markers or material within the foam segment. The foam expands into the target and engages the target from within.

The target is then dragged from the occluded vessel, thus restoring blood flow to the distal distribution of this vessel. Under fluoroscopic guidance, the retrieval device with its captured target is carefully withdrawn into the aforementioned cervical artery and into the guiding catheter. If an inflated occlusive balloon tip is used, it ensures retrograde blood flow within the cervical vessel (i.e., toward the guiding catheter tip), thus facilitating successful removal of the retrieval device and the trapped target.

### EXAMPLE 2

A device in accordance with one embodiment of the present invention shown in FIGS. 4A-4D is used. The device has two isolated formations of a hydrogel attached to a wire. Under fluoroscopic and/or digital roadmap imaging, the device is advanced into a lumen containing a matter to be removed. The device is maneuvered around the matter so that the matter is located between two formations. The device is left in place for several minutes. The hydrogel begins to swell with the absorption of ambient water so as to transition from a compressed into an expanded configuration. The matter becomes trapped between two expanded formations of the hydrogel and is dragged from the occluded vessel, thus restoring blood flow to the distal distribution of this vessel.

It will be apparent to those skilled in the art that various modifications and variations can be made in the system and methods of the present invention without departing from the spirit or scope of the invention. Thus, it is intended that the present invention cover modifications and variations of this invention that come within the scope of the appended claims and their equivalents.

1. A device for removing a matter from a body cavity of a patient, the device comprising:
an elongated carrier having a distal portion adapted for positioning inside the body cavity and a proximate portion; and
a radially expandable polymer circumferentially attached to the distal portion of the carrier and adapted to enter the matter while in a compressed configuration and capable of transitioning to an expanded configuration while inside the matter to penetrate and engage it from within.

2. The device of claim 1, wherein the polymer is a shape-memory polymer selected from a group consisting of polyurethane, polyethylene, polyethylene terephthalate, polyisoprene, styrene-butadiene copolymers and other styrenes, copolyester, ethylene-vinylacetate and other ethylene copolymers, polyacrylamide gel, polyacrylic acid and other acrylates, norbornane, polystyrene, and polynorbornene.

3. The device of claim 1, wherein the transition of the polymer between the compressed configuration and the expanded configuration is triggered by a physiological stimulus, an external stimulus, by a mechanical device, or their combination.

4. The device of claim 3, wherein the physiological stimulus is selected from a group consisting of body temperature, blood pH, an ion concentration in blood, and blood composition.

5. The device of claim 3, wherein the external stimulus is selected from a group consisting of solutions, the introduction of which into the blood vessel causes changes in the local chemical environment, changes in external temperature, light, magnetic field, ultrasound, radiation, and electrical field.

6. The device of claim 3 further comprising a delivery device adapted for positioning inside the cavity and having an internal lumen, wherein the distal portion of the carrier is slidably positioned within the lumen of the delivery device, wherein the polymer remains in the compressed configuration inside the delivery device and the polymer transitions into the expanded configuration when it exits the delivery device.

7. The device of claim 1, wherein the polymer comprises a predetermined amount of a therapeutic agent.

8. The device of claim 7, wherein the therapeutic agent is released when the polymer transitions to the expanded configuration.

9. The device of claim 7, wherein the therapeutic agent is selected from a group consisting of anti-thrombogenic, thrombolytic, anti-proliferative, anti-spasmodic, anti-coagulant, anti-platelet adhesion drugs, endothelial cells, and gene vectors.

10. The device of claim 1, wherein said polymer is a hydrogel or a foam.

11. The device of claim 10, wherein the hydrogel is selected from a group consisting of polyethylene oxide, polyvinyl alcohol, polyvinylpyrrolidone, polyhydroxyethyl methacrylate, polyetherpolycarbonatecollagen and polysaccharides.

12. The device of claim 10, wherein the polymer is the hydrogel and the transition of the hydrogel into the expanded configuration is triggered by hydration of the hydrogel or by application of a triggering fluid to the hydrogel.

13. The device of claim 11, wherein the triggering fluid has a predetermined pH or a composition that causes the hydrogel to transition into the expanded configuration.

14. The device of claim 10, wherein said foam is a shape-memory foam selected from a group consisting of polyurethane, a cross-linked ethylene-vinyl acetate, and polyethylene copolymers.

15. The device of claim 10, wherein said foam is a shape-memory foam having an original expanded configuration that can be compressed at a temperature above a glass transition temperature, Tg, to form the compressed configuration, wherein the foam retains its compressed configuration at a temperature below the Tg but returns substantially to its original expanded configuration when it is exposed to a temperature above the Tg.

16. The device of claim 15, wherein the Tg is above a body temperature and the device further comprises an external source of heat.

17. The device of claim 16, wherein the external source of heat is selected from a group consisting of electrical resistance, inductive, optical, and convective heating elements.

18. The device of claim 17, wherein the electrical resistance element comprises a metal coil or a semiconductive plastic that is circumferentially attached to the distal portion of the carrier and electrically connected to a controller through an insulated pathway, the controller capable of adjusting a voltage applied to the resistance element to maintain a predetermined temperature.

19. The device of claim 15, wherein the Tg is below a body temperature and the foam spontaneously transitions into the expanded configuration after being exposed to the body temperature for a predetermined time.

20. The device of claim 19, wherein the distal portion of the elongated carrier further comprises a steerable tip.

21. The device of claim 20, wherein the steerable tip is a shape-able platinum or stainless steel wire.

22. The device of claim 1, wherein the carrier is a guidewire or a catheter.

23. The device of claim 1, wherein the carrier has a sufficient length to serve as an exchange guidewire to deliver a secondary device over the carrier downstream from the expandable polymer.

24. The device of claim 23, wherein the secondary device is selected from the group consisting of catheters, angioplasty balloons, stents, and microcatheters.

25. The device of claim 1, wherein the expanded configuration has a general shape selected from a group consisting of a coil, a twisted ribbon, a screwlike structure, a disk, a sphere, a parachutelike structure, a formation comprising a plurality of ridges and troughs, and a formation comprising a plurality of outwardly extending spears.

26. The device of claim 1 or claim 25 comprising at least two isolated formations of the radially expandable polymer attached to the distal portion of the carrier.

27. The device of claim 26 comprising a plurality of progressively decreasing in size formations of the radially expandable polymer.

28. The device of claim 25, wherein the expanded configuration has the parachutelike structure surrounding and attached to the carrier, the parachutelike structure comprises a basket portion for collecting the matter and at least two supporting struts, wherein the basket portion has an opening facing the proximate portion of the carrier.

29. The device of claim 28, wherein the struts are reinforced by embedded wires or polymer fibers.

30. The device of claim 28, wherein the basket portion is made of a foam material.

31. The device of claim 25, wherein the expanded configuration has the twisted ribbon shape, wherein the polymer is capable of storing torque energy when in compressed configuration and releasing it in the expanded configuration.

32. The device of claim 31 further comprising a microdevice attached to the distal end of the carrier and an amount of the released energy is sufficient to drive the microdevice for a predetermined time.

33. The device of claim 32, wherein the microdevice dissolves fragments or traps clots, athermatous plaques, or debris in the body cavity.

34. The device of claim 31 wherein the polymer is a heat-activated memory-shape polymer, and the torque energy is applied by twisting the ribbon at a temperature above a Tg of the polymer, the polymer is stabilized by cooling down the ribbon, and the torque energy is released by heating the polymer above Tg.

35. The device of claim 1, wherein the body cavity is naturally existing or surgically created.

36. The device of claim 35, wherein the body cavity is selected from a group consisting of lumens of blood vessels, lumens of the alimentary tract, lumens of the genitourinary system, ventricles and cisterns of the brain, urinary bladder, cysts, vagina, uterine cavity, pseudocysts, abcesses, and fistulae.

37. The device of claim 1 further comprising a polymer coating applied to the radially expandable polymer.

38. The device of claim 37, wherein the polymer coating is capable of preventing fragmentation of the expandable polymer.

39. The device of claim 37, wherein the body cavity is a blood vessel lumen, the polymer coating carries a positive charge, a negative charge, or both, and wherein the coating is capable of attracting or repelling blood clots.

40. The device of claim 1 wherein the polymer provides a surface that carries a magnetic field, a positive charge, a negative charge, or their combination.

41. The device of claim 40, wherein the charge or magnetic field is an intrinsic property of the polymer, produced by chemical modification of the polymer's surface, or induced by application of external energy.

42. The device of claim 1, wherein the matter is a clot, an embolus, calculus, atherosclerotic plaque, loose tissue or neoplasm, inspissated fluid or secretion, or a foreign body.

43. The device of claim 1, wherein the compressed configuration of the radially expandable polymer has at least one feature imparting a flexibility to the polymer in the compressed configuration, improving the ability of the polymer to expand, or both.

44. The device of claim 43, wherein the feature is selected from a group consisting of cuts, slots, cells, cavities, holes, and indents.

45. The device of claim 44 comprising a plurality of cuts or slots oriented perpendicularly to a long axis of the carrier along an entire length of the expandable polymer, wherein each slot or cut is offset circumferentially from an immediately preceding slot or cut.

46. The device of claim 44 comprising a spiral cut.

47. The device of claim 1, wherein the radially expandable polymer exhibits an adhesive property to the matter.

48. The device of claim 1 further comprising a retrieval element adapted for positioning inside the body cavity, wherein the retrieval element has a proximal and a distal end, wherein the distal end comprises a self-deploying expandable sleeve, and wherein the retrieval element has a channel that extends through the entire length of the retrieval element and the expandable sleeve, wherein the distal portion of the carrier is sidably positioned within and adapted to move through the channel into the body cavity, and wherein the expandable polymer in its expanded configuration is capable of being at least partially retrieved into the expandable sleeve.

49. The device of claim 48 further comprising a pushing-pulling device operatively connected to the sleeve for deployment and retraction of the sleeve.

50. The device of claim 48, wherein the expandable sleeve is a balloon-expandable sleeve.

51. The device of claim 1 further comprising a wire coil running through the expandable polymer, the wire coil having a first end fixedly attached to the distal portion of the carrier and a second end movably attached to the proximate portion of the carrier, the device further comprising a barrier circumferentially attached to the carrier, wherein the barrier prevents movement of the second end when the carrier is pulled back, whereby the coil radially expands.

52. A method of retrieving a matter from a body cavity of a patient comprising:
(a) providing the device of claim 1;
(b) positioning the device inside the body cavity;
(c) allowing sufficient time for the expandable polymer to expand and engage the matter from within; and
(d) removing the device, whereby removing the matter.

53. A device for retrieving a matter from a body cavity of a patient, the device comprising:
an elongated carrier having a distal portion adapted for positioning inside the body cavity; and
at least two isolated formations of radially expandable polymer attached to the distal portion of the carrier, wherein each formation encloses the entire circumference of the carrier and wherein the formations are adapted to move through or around the matter while having a compressed configuration and capable of transitioning to an expanded configuration to trap the matter there between.

54. The device of claim 53, wherein said polymer is a hydrogel or a foam.

55. The device of claim 54, wherein said foam is a shape-memory foam having an original expanded configuration that can be compressed at a temperature above a glass transition temperature, Tg, to form the compressed configuration, wherein the foam retains its compressed configuration at a temperature below the Tg but returns substantially to its original expanded configuration when it is exposed to a temperature above the Tg.

56. The device of claim 55, wherein the polymer is the hydrogel and the transition of the hydrogel into the expanded configuration is triggered by a hydration of the hydrogel or by application of a triggering fluid to the hydrogel.

57. The device of claim 52 comprising a plurality of progressively decreasing in size formations of the radially expandable polymer.

58. The device of claim 53, wherein the expanded configuration has a general shape selected from a group consisting of a disk, a sphere, a formation comprising a plurality of ridges and troughs, and an outwardly extending spear.

59. The device of claim 53 further comprising a retrieval element adapted for positioning inside the body cavity, wherein the retrieval element has a proximal and a distal end, wherein the distal end comprises a self-deploying expandable sleeve and wherein the retrieval element has a channel that extends through an entire length of the retrieval element and the expandable sleeve, wherein the distal portion of the carrier is slidably positioned within and adapted to move through the channel into the body cavity, and wherein the isolated formations in their expanded configuration are capable of being at least partially retrieved into the expandable sleeve.

60. A method of retrieving a matter from a body cavity of a patient, the method comprising:
(a) providing the device of claim 52;
(b) positioning the device inside the body cavity;
(c) passing at least one formation through or around the matter;
(d) allowing sufficient time for the expandable polymer to transition from the initial compressed configuration to an expanded configuration, whereby trapping the matter between the formations; and
(e) removing the device.

61. A device for retrieving a matter from a body cavity of a patient, the device comprising:
an elongated carrier having a distal portion adapted to be positioned inside the body cavity; and
a radially expandable polymer circumferentially attached to the distal portion of the carrier and adapted to move through the matter while having a compressed configuration and capable of transitioning to an expanded configuration to engage the matter for retrieval from the body cavity, wherein the transition of the polymer is triggered by a physiological stimulus.

62. The device of claim 61, wherein the physiological stimulus is selected from a group consisting of body temperature, blood pH, an ion concentration in blood, and blood composition.

63. The device of claim 61, wherein said polymer is a hydrogel or a foam.

64. The device of claim 61, wherein the polymer is the hydrogel and the transition of the hydrogel into the expanded configuration is triggered by a hydration of the hydrogel inside the blood vessel.

65. The device of claim 61, wherein the expanded configuration has a general shape selected from a group consisting of a coil, a twisted ribbon, a screwlike structure, a disk, a sphere, a parachutelike structure, a formation comprising a plurality of ridges and troughs, and a formation comprising a plurality of outwardly extending spears.

66. The device of claim 61 further comprising a retrieval element adapted for positioning inside the body cavity, wherein the retrieval element has a proximal and a distal end, wherein the distal end comprises a self-deploying expandable sleeve and wherein the retrieval element has a channel that extends through an entire length of the retrieval element and the expandable sleeve, wherein the distal portion of the carrier is slidably positioned within and adapted to move through the channel into the body cavity, and wherein the expandable polymer in its expanded configuration is capable of being at least partially retrieved into the expandable sleeve.

67. A method of retrieving a matter from a body cavity of a patient, the method comprising:
(a) providing the device of claim 60;
(b) positioning the device inside the body cavity;
(c) allowing sufficient time for a physiological stimulus to act on the expandable polymer to cause its transition from the initial compressed configuration to an expanded configuration, thereby engaging the matter in a way that allows its removal; and
(d) retrieving the device, thereby removing the matter.

68. A device for retrieving a matter from a body cavity of a patient, the device comprising:
a retrieval element adapted for positioning inside the body cavity, wherein the retrieval element has a proximal end and a distal end, wherein the distal end comprises an expandable sleeve, wherein the retrieval element has a channel that extends through the entire length of the retrieval element and the expandable sleeve, the retrieval element further comprising an inflatable balloon positioned concentrically inside the channel, the balloon, when inflated, is capable of radially expanding the expandable sleeve;
an elongated carrier slidably positioned within the channel of the retrieval element, wherein the elongated carrier has a distal portion adapted to move through the expandable sleeve of the retrieval element and into the body cavity; and
a radially expandable polymer circumferentially attached to the distal portion of the carrier and adapted to move through or around the matter while having a compressed configuration and capable of transitioning to an expanded configuration to engage the matter, wherein the expandable polymer in its expanded configuration is capable of being at least partially retrieved into the expanded sleeve.

69. The device of claim 68, wherein the balloon is maintained in a deflated state during insertion of the retrieval element into the body cavity and is in an inflated state when the retrieval element is positioned in a desired location within the cavity.

70. The device of claim 68, wherein the carrier is slidably positioned through the balloon.

71. A method of retrieving a matter from a body cavity of a patient, the method comprising:
(a) providing the device of claim 68;
(b) positioning the retrieval element into the body cavity;
(c) advancing the distal portion of the carrier through the lumen of the retrieval element and the expandable sleeve of the retrieval element lumen into the blood vessel;
(d) moving the distal portion of the carrier through or around the matter;
(e) allowing sufficient time for the expandable polymer to transition from the initial compressed configuration to an expanded configuration, whereby trapping the matter;
(f) inflating the balloon, whereby expanding the sleeve; and
(g) retrieving, at least partially, the carrier with the expandable polymer in its expanded configuration into the expanded sleeve.

72. A method of retrieving a matter from a body cavity of a patient, the method comprising:
(a) providing a device having a carrier with a distal portion and a radially expandable polymer circumferentially attached to the distal portion of the carrier, wherein the expandable polymer has an initial compressed configuration;
(b) positioning the distal portion of the carrier inside the body cavity;
(c) positioning the expandable polymer inside the matter;
(d) allowing sufficient time for the expandable polymer to transition from the initial compressed configuration to an expanded configuration, whereby penetrating and engaging the matter from within; and
(e) retrieving the device from the body cavity, thereby removing the matter.

73. The method of claim 72, wherein the polymer is a hydrogel and step (d) further comprises applying a triggering fluid to the hydrogel to cause its transition into the expanded configuration.

74. The method of claim 72, wherein the polymer is a foam and step (d) further comprises applying an external stimulus to cause its transition into the expanded configuration.

75. The method of claim 72, wherein the device further comprises a retrieval element adapted for positioning inside the body cavity, wherein the retrieval element has a proximal and a distal end, wherein the distal end comprises a self-deploying expandable sleeve, and wherein the retrieval element has a channel that extends through the entire length of the retrieval element and the expandable sleeve, wherein the distal portion of the carrier is slidably positioned within and adapted to move through the channel into the body cavity, the method further comprising a step of retrieving, at least partially, the carrier with the expandable polymer in its expanded configuration into the expandable sleeve before the step of retrieving the device from the body cavity.

76. A method of localized delivery of a therapeutic agent, the method comprising: (a) providing a removable device having a carrier with a distal portion and a radially expandable polymer circumferentially attached to the distal portion of the carrier, wherein the expandable polymer has an initial compressed configuration;
(b) advancing the distal portion of the carrier to a site in a body; and
(c) allowing sufficient time for the expandable polymer to transition from the initial compressed configuration to an expanded configuration delivering the therapeutic agent.

77. The method of claim 76, wherein the site is located in a lumen, a cavity, or a solid tissue of the body.

78. The method of claim 76, wherein step (c) further comprises applying an external or a physiological stimulus to the polymer to cause its expansion.

79. The method of claim 78, wherein the physiological stimulus is selected from a group consisting of body temperature, blood pH, an ion concentration in blood, and blood composition.

80. The method of claim 78, wherein the external stimulus is selected from a group consisting of solutions, the introduction of which into the blood vessel causes changes in the local chemical environment, changes in external temperature, light, magnetic field, ultrasound, radiation, and electrical field.

81. The method of claim 76, wherein the therapeutic agent is selected from a group consisting of anti-thrombogenic, thrombolytic, anti-proliferative, anti-spasmodic, anti-coagulant, anti-platelet adhesion drugs, endothelial cells, drugs encased in nanoshells, and gene vectors.

82. The method of claim 81, wherein the thrombolytic drug is selected from a group consisting of tissue plasminogen activator (tPA), streptokinase, a calcium ion influx inhibitor, urokinase, and their analogs.

83. The method of claim 76, wherein the site comprises a lumen of a blood vessel containing a matter obstructing the lumen, wherein step (b) further comprises positioning expandable polymer inside the matter and step (c) comprises delivering the therapeutic agent from within the matter.

84. A method of retrieving a matter from a body cavity of a patient, the method comprising:
(a) providing a device having a carrier with a distal portion and at least two isolated formations of radially expandable polymer attached to the distal portion of the carrier, wherein each formation encloses the entire circumference of the carrier and the expandable polymer has an initial compressed configuration;
(b) positioning the distal portion of the carrier inside the body cavity;
(c) passing at least one formation through or around the matter;
(d) allowing sufficient time for the expandable polymer to transition from the initial compressed configuration to an expanded configuration, whereby trapping the matter between the formations; and
(e) retrieving the device, thereby removing the matter.

85. A method of retrieving a matter from a body cavity of a patient, the method comprising:
(a) providing a device having a radially expandable polymer circumferentially attached to the distal portion of the carrier, wherein the expandable polymer has an initial compressed configuration;
(b) positioning the distal portion of the carrier inside the body cavity, through or around the matter;
(c) allowing a sufficient time for a physiological stimulus to act on the expandable polymer to cause its transition from the initial compressed configuration to an expanded configuration, whereby engaging the matter in a way that allows its removal; and
(d) retrieving the device, thereby removing the matter.

86. The method of claim 85, wherein the physiological stimulus is selected from a group consisting of body temperature, blood pH, an ion concentration in blood, and blood composition.

87. A method of retrieving a matter from a body cavity of a patient, the method comprising:
(a) providing a device comprising:
a retrieval element adapted for positioning inside the body cavity, wherein the retrieval element has a proximal and a distal end, wherein the distal end comprises an expandable sleeve, wherein the retrieval element has a channel that extends through the entire length of the retrieval element and the expandable sleeve, the retrieval element further comprising an inflatable balloon positioned concentrically inside the channel, and the balloon, when inflated, is capable of radially expanding the expandable sleeve;
an elongated carrier slidably positioned within the channel of the retrieval element, the elongated carrier having a distal portion; and
a radially expandable polymer circumferentially attached to the distal portion of the carrier, wherein the expandable polymer has an initial compressed configuration;
(b) positioning the retrieval element inside the body cavity;
(c) advancing the distal portion of the carrier through the channel of the retrieval element and the expandable sleeve of the retrieval element into the body cavity;
(d) moving the distal portion of the carrier through or around the matter;
(e) allowing sufficient time for the expandable polymer to transition from the initial compressed configuration to an expanded configuration, whereby trapping the matter;
(f) inflating the balloon, whereby expanding the sleeve; and
(g) retrieving, at least partially, the carrier with the expandable polymer in its expanded configuration into the expanded sleeve.
